(19) United States
(12) Patent Application Publication
(10) Pub. No.: US 2008/0249501 A1
(43) Pub. Date: Oct. 9, 2008

(54) Methods for Simultaneous Injection and Aspiration of Fluids During a Medical Procedure

(75) Inventor: Dwayne S. Yamasaki, St. Augustine, FL (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/058,247

(22) Filed: Mar. 28, 2008

Related U.S. Application Data
(63) Continuation of application No. 60/910,770, filed on Apr. 9, 2007.

(51) Int. Cl. A61M 31/00

(52) U.S. Cl. 604/506

(57) ABSTRACT

Methods for simultaneous injection and aspiration of fluids during a medical procedure are disclosed. Embodiments include methods for operating medical devices within the subarachnoid space of the spinal column to gain access to the ventricles of the brain, as well as the surrounding cranial subarachnoid space. A dual lumen constant volume aspiration catheter is disclosed that injects a volume of injectable fluid to break up an obstruction within the brain or cranial subarachnoid space while simultaneously aspirating a same volume of aspirated fluid from the treatment site. Methods hereof include constant volume re-circulation of cerebral spinal fluid to and from a treatment area within one of the brain and the surrounding cranial subarachnoid space, which may be desirable during a ventriculostomy.

### CROSS REFERENCE TO RELATED APPLICATIONS

This application claims the benefit under 35 U.S.C §119(e) of U.S. Appl. No. 60/910,770 filed Apr. 9, 2007, which is incorporated by reference herein in its entirety.

### FIELD OF THE INVENTION

The invention relates generally to methods for use during surgical procedures. More specifically, the invention is related to methods for use in simultaneously injecting and removing fluids at the site of an obstruction within the brain.

### BACKGROUND OF THE INVENTION

Hydrocephalus is sometimes referred to as ‘water on the brain’. A watery fluid, known as cerebrospinal fluid or CSF, is produced continuously inside each of the four spaces or ventricles inside the brain. The CSF normally flows through narrow pathways from one ventricle to the next, then out across the outside of the brain and down the spinal cord. The CSF is absorbed into the bloodstream and re-circulates. The amount and pressure of CSF in the brain are normally kept within a fairly narrow range. However, if the drainage pathways are blocked at any point, the fluid accumulates in the ventricles inside the brain, or in the subarachnoid space causing them to swell, which thereby results in increased intracranial pressure and compression of the surrounding tissue. In babies and infants, hydrocephalus will cause the head to enlarge. In older children and adults, the head size cannot increase as the bones which form the skull are completely joined together and, as such, hydrocephalus may cause severe headaches, nausea, abnormal gait, dementia and/or permanent brain damage.

Hydrocephalus is often treated by insertion of a drainage system, e.g., a ventriculoperitoneal (VP) shunt. A shunt is simply a drain that diverts or “shunts” the accumulated CSF from the obstructed drainage pathways and returns it to the bloodstream. Symptoms caused by raised pressure usually improve after successful shunting, but some problems will remain. Recently neuroendoscopy, or telescopic surgery, makes treatment of hydrocephalus in some patients possible without shunting, the success rate depending on the etiology of the hydrocephalus. Management of hydrocephalus by endoscopic third ventriculostomy (ETV) involves creating an opening in the floor of the third ventricle, allowing the CSF to bypass the obstruction. This is a procedure that does not have the complications of shunt insertion, i.e., infection is rare and morbidity is very low. However, placement of a VP shunt and ETV procedures require burr holes in the skull and introduction of medical apparatus through the cerebral cortex and underlying white matter.

Slit ventricles are usually caused by excess removal of CSF, resulting in collapsing of the ventricles. Sometimes they are so small that they are barely visible on CT scan or MRI. Slit ventricles can occur after severe head injury or viral infection of the brain. In both conditions, the brain becomes so swollen that the fluid is pushed out of the ventricles. Slit ventricles may also occur after cerebrospinal fluid diversion, for example due to placement of a VP shunt or during/after certain surgical procedures. Slit ventricle syndrome is not a single pathologic entity, but is a symptom complex with several etiologies. For example, slit ventricle syndrome can appear in patients with a functioning shunt and in whom the brain has lost part of its elasticity. The symptoms consist, inter alia, of headaches, vomiting, and drowsiness.

The withdrawal of fluid from the brain that may occur during shunt placement/retrieval, ETV procedures and various other surgical procedures may also result in slit ventricle syndrome due to over drainage of fluids or other adverse consequences, if there is an under drainage of fluids. What is needed are methods that maintain the cerebrospinal fluid volume and/or pressure within a narrow range when performing brain surgeries and therapies in order to prevent under or over drainage of CSF and the attendant consequences thereof.

### BRIEF SUMMARY OF THE INVENTION

Embodiments hereof disclose methods for simultaneous injection and aspiration of fluids, such as a cerebral spinal fluid, during a medical procedure. Embodiments include methods for operating medical devices within the subarachnoid space of the spinal column to gain access to the ventricles of the brain, as well as the surrounding cranial subarachnoid space. A dual lumen constant volume aspiration catheter is disclosed that injects a volume of injectable fluid to break up an obstruction within the brain or cranial subarachnoid space while simultaneously aspirating a same volume of aspirated fluid, such as CSF, from the treatment site. Methods hereof also include constant volume re-circulation of cerebral spinal fluid to and from a treatment area within one of the brain and the surrounding cranial subarachnoid space, which may be performed during a ventriculostomy.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 illustrates portions of the anatomy of the brain.

FIG. 2 illustrates portions of the anatomy the lower spinal column with a catheter introducer and a catheter positioned within the subarachnoid space.

FIG. 3 illustrates a side view of an aspiration catheter according to an embodiment of the present invention.

FIG. 3A is a cross-sectional view taken along line A-A of FIG. 3.

FIG. 4 illustrates a cross-sectional view of an aspiration syringe according to an embodiment of the present invention.

FIG. 5 illustrates a cross-sectional view of a distal portion of the aspiration catheter of FIG. 3.

FIG. 6 illustrates a cross-sectional view of a distal portion of the aspiration catheter of FIG. 3 according to an alternate embodiment.

FIG. 7 illustrates a side view of an aspiration catheter according to another embodiment of the present invention.

FIG. 7A is a cross-sectional view taken along line A-A of FIG. 7.

FIG. 8 illustrates a side view of an aspiration catheter according to another embodiment of the present invention.

FIG. 9 illustrates a side view of an aspiration catheter according to another embodiment of the present invention.

### DETAILED DESCRIPTION OF EMBODIMENTS OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

Non-communicating hydrocephalus due to hemorrhage, tumors, fibrosing meningitis, edema, or other obstructions, such as infectious material, is primarily treated by ventriculoperitoneal (VP) shunts. These devices are catheters that are surgically lowered through the skull and brain to have one end positioned in the lateral ventricle. The other end of the catheter is tunneled under the skin and positioned in the peritoneal cavity of the abdomen, or right atrium of the heart, where the cerebrospinal fluid is absorbed or drained respectively. The failure rate for these devices ranges from 30% to 40% due to clogging of the catheter, infection, and/or faulty pressure or one-way valves. The procedure also requires burring holes in the skull and passage of the shunt through the cerebral cortex and underlying white matter, which may cause damage to those parts of the brain. An alternative treatment to this invasive procedure is disclosed below in accordance with various embodiments of the present invention. However, embodiments hereof also include using a constant volume device during ventriculostomy procedures to remove iatrogenic debris in the CSF, and improve efficacy of aspiration of debris by increased turbulence, i.e., mixing of hematoma or debris, while maintaining normal ventricular volume.

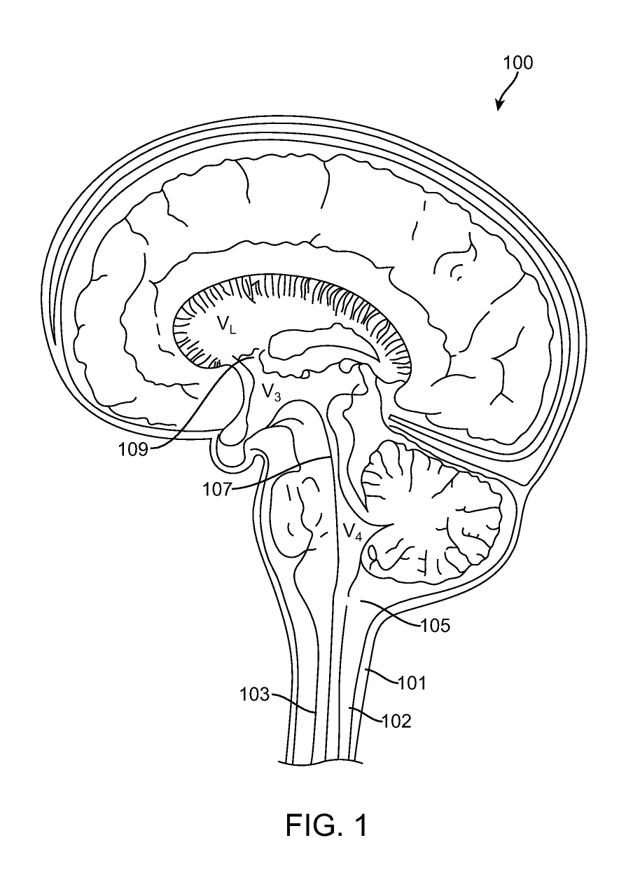

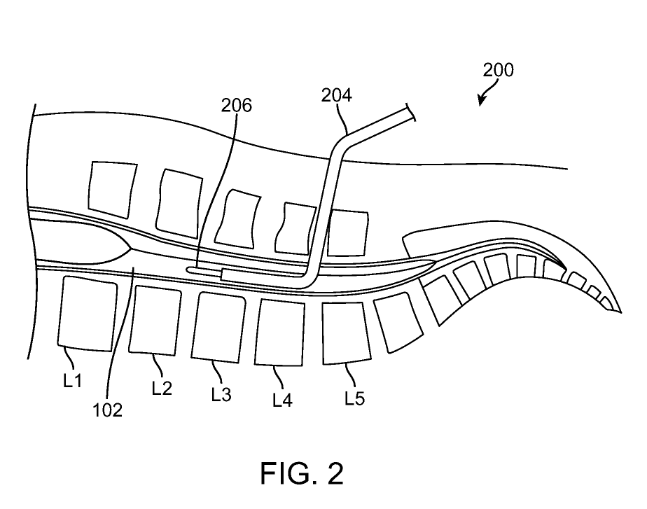

FIGS. 1 and 2 illustrate portions of the anatomy of the brain 100 and lower spinal column 200, respectively. Embodiments according to the present invention include medical apparatus and methods for breaking up and removing clots, hemorrhage or other obstructions within the brain that cause hydrocephalus via access through the subarachnoid space 102. Subarachnoid space 102 is an area between the arachnoid mater 101 and pia mater 103 of the spinal column that surrounds the body of the spinal cord and contains the cerebrospinal fluid. In an embodiment, an obstruction/clot within the ventricles of the brain may be accessed through the spinal subarachnoid space 102. Initially, a lumbar puncture may be performed at the L3-L4 or L4-L5 space or a cervical puncture, such that a catheter introducer 204, and/or a guiding catheter, may be positioned within subarachnoid space 102. A subarachnoid catheter 206, i.e., a steerable single or multi-lumen microcatheter, may then be inserted through catheter introducer 204 and into subarachnoid space 102. Subarachnoid catheter 206 may then be navigated up to the skull base within subarachnoid space 102, where it may then be steered through, for example, the foramen of Magendie 105 or one of the foramina of Luschka (not shown) and into the fourth ventricle V₄. Subarachnoid catheter 206 may then be navigated cephalad through the cerebral aqueduct 107 and into the third ventricle V₃ to the site of the obstruction. If the obstruction is deeper within the ventricles, subarachnoid catheter 206 may be positioned through the foramen of Monro 109, as needed, to access one of the lateral ventricle V_{L}.

The subarachnoid catheter can be navigated to the occlusion or hemorrhage site, for example, within the ventricles, interpeduncular cistern, etc. In addition, the subarachnoid catheter may be used to poke through or penetrate the septum pellucidum to treat asymmetric ventriculomegaly, drain an arachnoid cyst, and/or to allow drainage of excess CSF from the arachnoid cisterns.

Navigation of subarachnoid catheter 206 through subarachnoid space 102 and within the ventricles of the brain may be assisted with a guidewire. The position of the guidewire within the subarachnoid space and ventricles of the brain, as well as that of other medical devices used in accordance with methods herein, may be monitored by using any suitable imaging technology, such as magnetic resonance imaging, fluoroscopy, endoscopy, fiberoptic visualization, computed tomography, thermal imaging, sonography, X-ray visualization, and/or any combination of these. Accordingly, access to the ventricles via a lumbar or cervical puncture by methods according to the present invention significantly reduces recovery time to a day or sooner, instead of several days to weeks as is customary with the more invasive VP procedures currently in practice, which as mentioned may include burr holes through the skull and invasion of the cerebral cortex and subcortical white matter by medical apparatus.

If the non-communicating hydrocephalus is caused by a subarachnoid or intraventricular hemorrhage and clotting, or other obstruction, such as from infectious material, the obstruction may be slowly aspirated through subarachnoid catheter 206. Once the obstruction is removed, aspiration may continue until the excess cerebrospinal fluid within the ventricle is removed, with care being taken not to remove too much cerebrospinal fluid and causing slit ventricle syndrome. The placement of subarachnoid catheter 206 and removal of cerebrospinal fluid may be monitored by fluoroscopy.

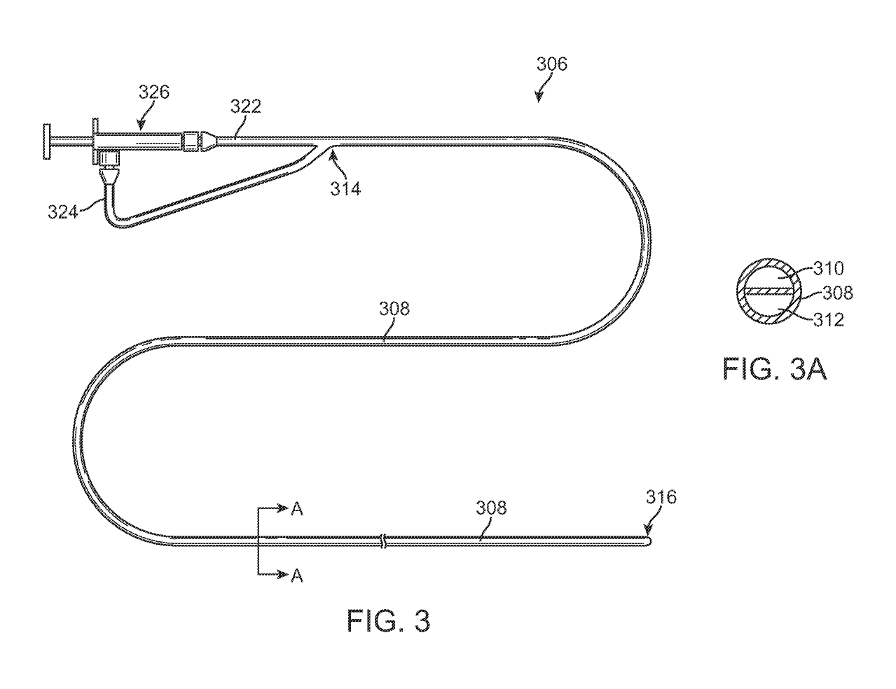

In another embodiment of the present invention shown in FIGS. 3-6, aspiration may be performed by a subarachnoid aspiration catheter 306 that simultaneously aspirates the obstruction and injects fluids to maintain the correct cerebrospinal fluid volume within the ventricles. Aspiration catheter 306 includes an elongate tubular member 308 that is flexible enough to navigate around, distribution vessels, spinal rootlets and cranial nerves found within subarachnoid space 102 while being longitudinally incompressible enough to be pushed therethrough. Tubular member 308 may be made of one or more suitable polymeric materials, including a thermoplastic material, such as polyethylene block amide copolymer, polyvinyl chloride, polyethylene, polyethylene terephthalate, polyamide, polyurethane and/or a thermoset polymer, such as polyimide.

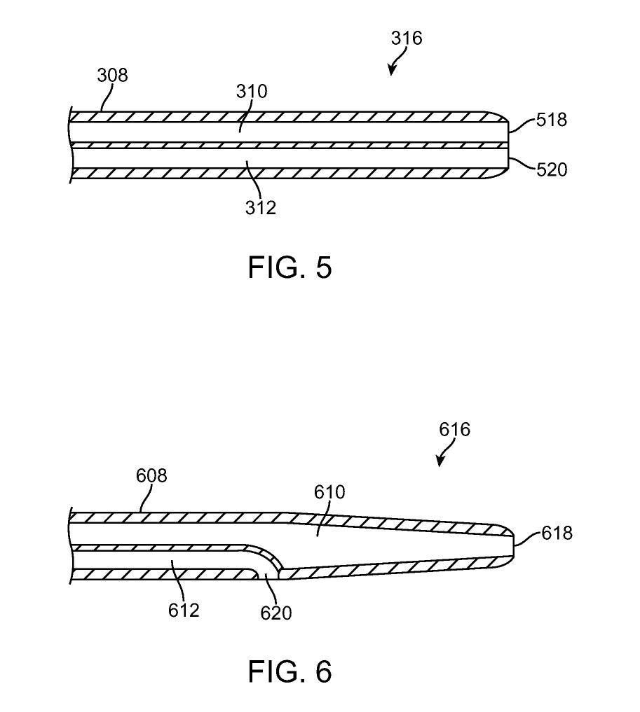

A fluid injection lumen 310 and an aspiration lumen 312 extend from a proximal end 314 to a distal end 316 of tubular member 308. With reference to FIG. 5, distal end 316 of tubular member 308 includes a distal port 518 of fluid injection lumen 310 in a side-by-side arrangement with a distal port 520 of aspiration lumen 312. In an alternate embodiment, distal end 616 of tubular member 608 includes distal port 618 of fluid injection lumen 610 that is disposed distal of distal port 620 of aspiration lumen 612, which exits from a side surface of tubular member 608. In an alternate embodiment (not shown), the fluid injection and aspiration lumens may be coaxial such that one of the lumen is annular and surrounds the other lumen. During operation, a medical fluid is forced from distal port 518 of fluid injection lumen 310 to break up the obstruction while debris and cerebrospinal fluid is aspirated via distal port 520 into aspiration lumen 312 for removal from the treatment site. In accordance with an embodiment of the present invention, the proximity of distal ports 518, 520 may be such that simultaneous injection and aspiration of fluids creates vortices or increased turbulence at the distal tip of catheter 306 that will help break up the obstruction.

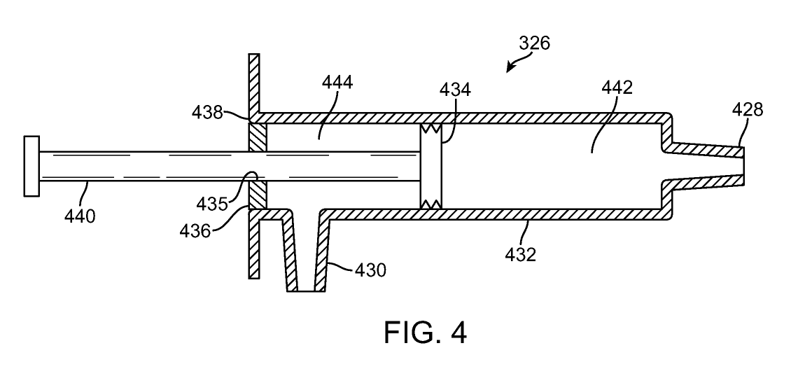

Each of fluid injection lumen 310 and aspiration lumen 312 has a proximal portion 322, 324, respectively, that is connectable to an aspiration syringe 326 via a luer-lock arrangement, or other such fluid fittings. As such, a proximal port of fluid injection lumen 310 is attached to a distal port 428 of aspiration syringe 326 and a proximal port of aspiration lumen 312 is attached to a proximal port 430 of aspiration syringe 326. As shown in FIG. 4, aspiration syringe 326 includes a cylindrical barrel 432 having a plunger 434 slidably disposed therein. An annular barrel seal 436 closes a proximal end 438 of barrel 432 and allows for sealed sliding movement of a plunger shaft 440 thorough a central opening 435. Distal and proximal syringe ports 428, 430 are positioned on either side of plunger 434, such that barrel 432 is divided into a distal chamber 442 for holding a medical fluid and a proximal chamber 444 for holding aspirated body fluid, e.g., cerebrospinal fluid. During operation, aspiration syringe 326 injects a saline or other suitable fluid from distal chamber 442 into fluid injection lumen 310 of aspiration catheter 306 while simultaneously, and at the same rate, drawing aspirated cerebrospinal fluid and debris into proximal chamber 444 from aspiration lumen 312 of aspiration catheter 306. In various embodiments, barrel 432 of aspiration syringe 326 is of a volume in the range of 10 cc to 60 cc.

If needed, aspiration catheter and syringe may adapted to allow aspiration of the obstruction or a constant exchange of CSF over a prolonged period of time by providing a medical fluid reservoir in fluid communication with the distal chamber 442 and an aspirated fluid reservoir in fluid communication with the proximal chamber 444, One-way valves and volume displacement mechanisms, either mechanical or computer controlled, may be used to maintain a constant volume of fluid into and out of the respective reservoirs in order to reduce the risk of removing too much cerebrospinal fluid from the treatment site.

Subarachnoid aspiration catheter 306 may be tracked to the treatment site via a guidewire that has been previously positioned within the subarachnoid space to and beyond the obstruction. In addition, any one of the imaging technologies previously mentioned may be used to aid in guiding the subarachnoid aspiration catheter 306 to the treatment site. In certain medical applications, another subarachnoid catheter may already be in-dwelling, such that a guidewire may be tracked through the lumen of the in-dwelling subarachnoid catheter to and through the obstruction with the subarachnoid catheter being subsequently removed. Aspiration catheter 306 may then be tracked along the guidewire up to the obstruction, with the guidewire being subsequently removed.

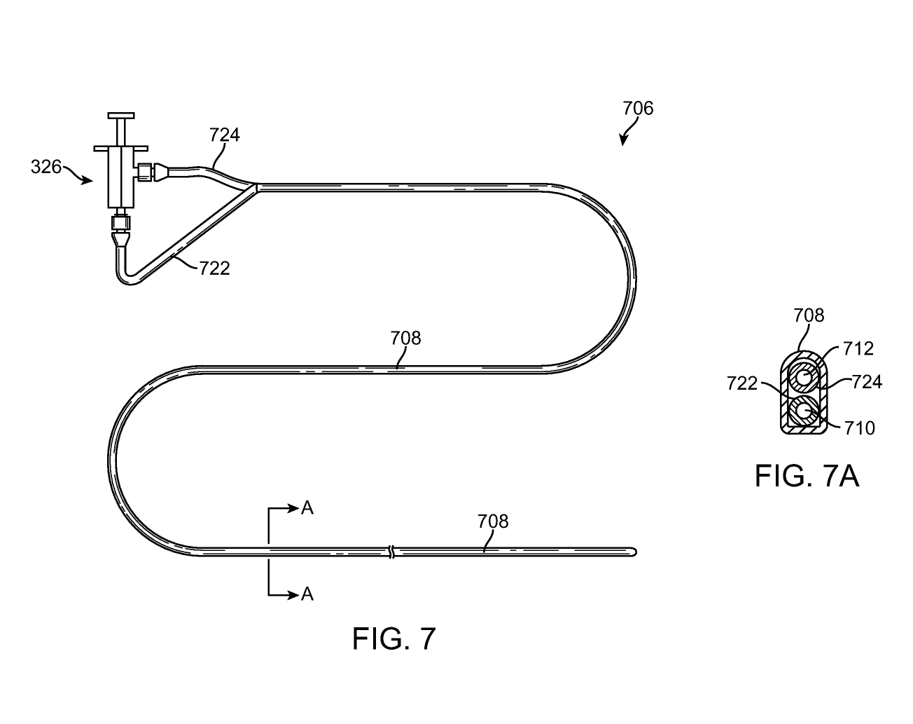

FIGS. 7 and 7A illustrate aspiration catheter 706 according to an alternate embodiment of the present invention. Aspiration catheter 706 includes a polymeric sheath 708 that encases a fluid injection shaft or tubing 722 with a fluid injection lumen 710 and an aspiration shaft or tubing 724 with an aspiration lumen 712. In the embodiment of FIG. 7, aspiration catheter 706 has an elliptical cross-section. However in another embodiment, aspiration catheter 706 may be configured to have a circular cross-section as shown in the embodiment of FIG. 3A or coaxial lumens. Polymeric sheath 708 and injection and aspiration shafts 722, 724 may be made of one or more suitable polymeric materials, including a thermoplastic material, such as polyethylene block amide copolymer, polyvinyl chloride, polyethylene, polyethylene terephthalate, polyamide, polyurethane and/or a thermoset polymer, such as polyimide, respectively. Aspiration syringe 326 is attached to aspiration catheter 706 to function in a similar manner as described with reference to the embodiment of FIG. 3.

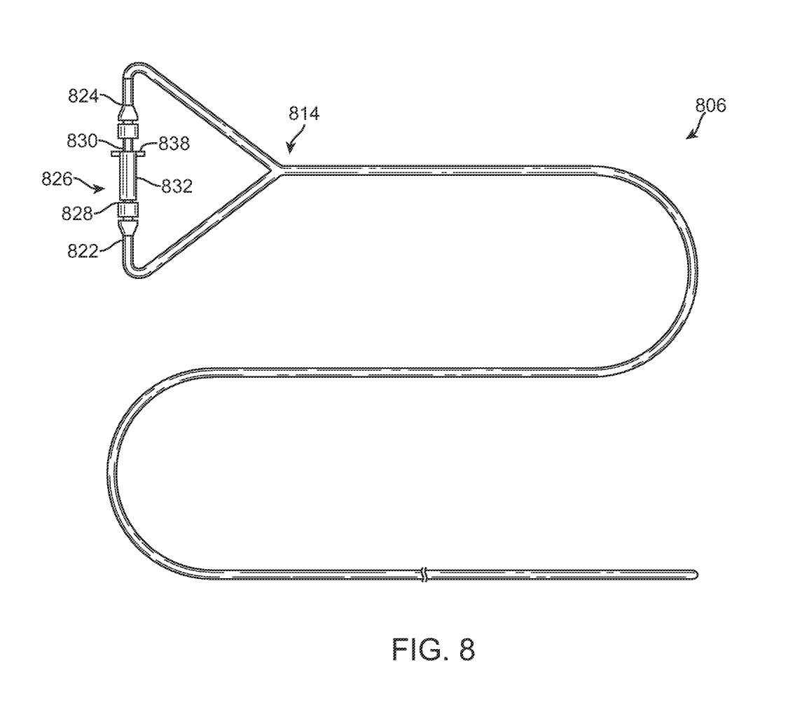

FIG. 8 illustrates aspiration catheter 806 according to an alternate embodiment of the present invention. Aspiration catheter 806 is of a dual lumen catheter construction, such as any of the catheter constructions previously described or as may be apparent to one of ordinary skill in the art, and includes an aspiration syringe 826 attached at proximal end 814 thereof. Aspiration syringe 826 includes distal port 828 attached to a proximal port 822 of the fluid injection lumen of aspiration catheter 806 and proximal port 830 attached to a proximal port 824 of the aspiration lumen of aspiration catheter 806. Proximal port 830 of aspiration syringe 826 extends from proximal end 838 of a barrel 832 of aspiration syringe 826.

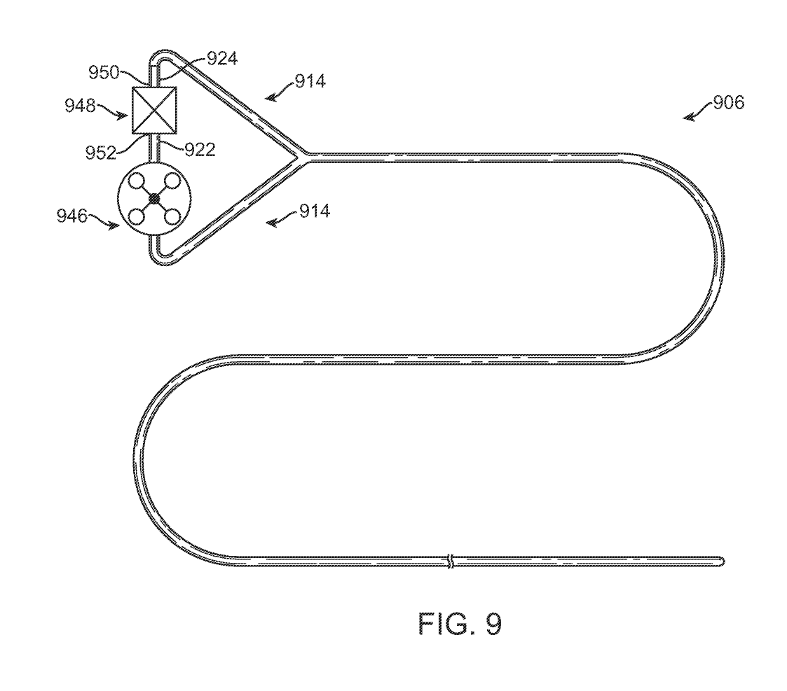

In another embodiment illustrated in FIG. 9, aspiration catheter 906 is of a dual lumen catheter construction, such as any of the catheter constructions previously described or as may be apparent to one of ordinary skill in the art, and utilizes a peristaltic pump 946 to move fluid therethrough. This embodiment may be particularly beneficial for use in a ventriculostomy procedure and in treatment of patients with normal pressure hydrocephalus (NPH). A peristaltic pump is a type of positive displacement pump used for pumping a fluid contained within a flexible tube fitted inside the pump casing. Peristaltic pumps are typically used in medical applications to pump clean or sterile fluids because the pumping mechanism does not contact and therefore cannot contaminate the fluid. A rotor with a number of rollers, shoes or wipers attached to the external circumference compresses the flexible tube as the rotor turns, such that the part of the tube under compression closes, or occludes, thus forcing the fluid to move through the tube. Additionally, the tube opens to its natural state after the passing of the cam, aka, restitution, and fluid flow is induced into the pump. Embodiments according to the present invention may utilize a lower pressure peristaltic pump, typically having a dry casing, rollers and non-reinforced tubing, which is sometimes referred to as a tube or tubing pump and/or a dual head peristaltic pump.

In a method according to an embodiment of the present invention, aspiration catheter 906 and peristaltic pump 946 may be beneficial for use during any ventriculostomy procedure, including an endoscopic third ventriculostomy, by removing cerebrospinal fluid from the treatment area, filtering the fluid and reintroducing the fluid within the brain at a constant rate. This method constitutes a “closed” system as the same CSF is removed from and then reintroduced back into the treatment area. Another CSF recirculation embodiment according to a method hereof may be used for NPH patients where the constant volume aspiration system is left in place chronically, i.e., beyond 24-48 hours, and the treatment area may be within the brain or in the subarachnoid space between the brain and the cranium. In an alternate open system embodiment, the aspirated fluid may be discarded and replaced with a saline or artificial CSF pumped from an external replacement fluid reservoir.

A proximal portion 914 of aspiration catheter 906 includes a continuous length of tubing that passes through the peristaltic pump to permit cerebrospinal fluid to be removed from the treatment site through the aspiration lumen of the aspiration catheter and subsequently reintroduced to the treatment site through the injection lumen of the aspiration catheter. Operation of peristaltic pump 946 permits the cerebrospinal fluid to be removed and reintroduced at a constant rate thereby avoiding any complications associated with an imbalance of cerebrospinal fluid during the procedure.

In embodiments where the CSF is recycled, a filter 948 may be employed upstream of peristaltic pump 946 to remove debris from the cerebrospinal fluid prior to its re-injection, or reintroduction, into the brain. In an embodiment where filter 948 is employed, a proximal end 924 of the aspiration lumen of aspiration catheter 906 is attached at a downstream end 950 of filter 948 and a proximal end 922 of the fluid injection lumen of aspiration catheter 906 is attached at an upstream end 952 of the filter. Peristaltic pump 946 may be placed upstream, as shown in FIG. 9, or may be placed downstream of filter 948 with the catheter tubing passing through peristaltic pump 946. Peristaltic pump 946 as used in embodiments of the present invention permits the continual or intermittent circulation of cerebrospinal fluid during a ventriculostomy procedure.

In a method according to another embodiment of the present invention, aspiration catheter 906 and peristaltic pump 946 may be beneficial for use as a treatment for chronic normal pressure hydrocephalus (NPH). Patients with NPH have a high correlation of developing Alzheimer's disease, and it is known that beta-amyloid protein is present in their CSF. Chronic or periodic replenishing of CSF in these patients could reduce the risk of Alzheimer's disease in these patients by removing the beta-amyloid proteins from their CSF.

Subarachnoid aspiration catheters in accordance with various embodiments of the present invention may be approximately 150 cm in length with a luer-lock attachment on each of its proximal ports. A distal tip of an aspiration catheter in accordance with various embodiments may include a rounded edge to minimize the likelihood of catching or tearing vessels, spinal nerve rootlets and central nervous system tissue as it is tracked to the cite of the obstruction within the brain ventricles. The distal tip of the aspiration catheter may also include a radiopaque marker to facilitate accurate positioning of the catheter by fluoroscopy. In various embodiments, a distal portion of the aspiration catheter may have a diameter ranging from 3F to 9F depending on the application in which it is to be used.

In a method according to an embodiment of the present invention, a clinician determines that a lumbar or cervical puncture may be performed without the risk of cerebral herniation. The lumbar puncture is performed with a catheter introducer, or another appropriate medical instrument, and a Touhy Borst valve is attached. The aspiration catheter is inserted through the Touhy Borst valve and catheter introducer to thereby gain access to the spinal subarachnoid space. The aspiration catheter is then navigated superiorly within the spinal subarachnoid space to the base of the skull. Navigation of the aspiration catheter may be assisted by any one of the imaging technologies mentioned above. The aspiration catheter may then make entry into the fourth ventricle through the foramen of Magendie or one of the foramina of Luschka or may remain within the cranial subarachnoid space. The aspiration catheter is then tracked cranially within the ventricles or cranial subarachnoid space until the distal tip is positioned proximate the obstruction, which may be a blood clot, hemorrhage or other obstructive matter. Radiopaque markers may be used to aid in positioning of the distal tip of the catheter.

A dual-head peristaltic pump is then used to inject a volume of a fluid, such as saline, through the fluid injection lumen of the aspiration catheter and out of the distal injection port to break up the obstruction. Concurrently, the peristaltic pump draws a same volume of cerebrospinal fluid into the aspiration lumen of the aspiration catheter by a suitable vacuum created therein. In this manner, a constant volume and rate of fluid injection and aspiration is maintained at the surgical site to prevent a detrimental imbalance of pressure or over/under drainage of the cerebrospinal fluid from the area. Upon ablation of the obstruction, the aspiration catheter is withdrawn. In another embodiment, a constant-volume piston actuator pump can be used to generate pulsatile injections to help break up the clot while maintaining normal CSF volume within the subarachnoid space and/or ventricles.

In another embodiment of the present invention, the clot or obstruction may be initially broken up or loosened by an ultrasonic guidewire. The structure of a blood clot caused by subarachnoid or intraventricular hemorrhage may not necessarily form such that the clot is readily susceptible to aspiration. In some cases, blood clotting will occur along the ventricular walls making aspiration difficult. In addition, other types of obstructions that may cause hydrocephalus, such as infectious material and necrotic debris, may be difficult to aspirate without pretreatment. In such presentations where a clot or obstruction cannot be easily removed by aspiration alone, the clot or obstruction may be initially treated, i.e., loosened and/or broken up, through the use of a guidewire that generates ultrasonic waves in the cerebrospinal fluid. The ultrasonic guidewire is similar to an OmniSonics wire, or may be used in conjunction with that device. An ultrasonic guidewire according to an embodiment of the present invention may be tracked to the obstruction within the lumen of an in-place subarachnoid catheter and then activated to break up the obstruction while the debris is aspirated through the catheter.

In another embodiment of the present invention, an aspiration catheter for use herein may include a compliant balloon positioned proximal of the distal ports, such as fluid injection port 518 in FIG. 5 and fluid aspiration port 620 in FIG. 6. The inflated balloon will conform to the treatment site within the subarachnoid space and may facilitate aspiration of the obstruction instead of primarily removing CSF so that the aspiration is more effective in removing the obstruction. In another embodiment, use of a compliant balloon to temporarily occlude the treatment site also allows treatment of a blood clot by injecting rt-PA into the obstruction via a lumen of the subarachnoid catheter with less diffusion, thereby improving localized delivery of rt-PA.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A method of breaking up and removing an obstruction that causes non-communicating hydrocephalus, comprising the steps of:
providing a constant volume aspiration catheter;
gaining access to the subarachnoid space of the spinal column;
navigating the aspiration catheter through the subarachnoid space to the base of the skull;
navigating the aspiration catheter along the brain in one of the subarachnoid space and ventricles until an obstruction for treatment is reached;
positioning a distal tip of the aspiration catheter proximate the obstruction to be treated; and
injecting a volume of a fluid from a first lumen of the aspiration catheter out of a distal opening of the first lumen to break up the obstruction, while concurrently drawing a same volume of aspirated fluid into a distal opening of a second lumen of the aspiration catheter to maintain a constant volume of fluid within the brain.

2. The method of claim 1, wherein the steps of navigating the aspiration catheter include entering the fourth ventricle of the brain via one of the foramen of Magendie and the foramina of Luschka and navigating through the ventricles of the brain until the obstruction is reached.

3. The method of claim 1, wherein the steps of navigating the aspiration catheter include navigating the aspiration catheter in the subarachnoid space proximate the brain to a hemorrhage site, wherein the hemorrhage site is the obstruction to be treated.

4. The method of claim 1, wherein the step of gaining access to the subarachnoid space includes a lumbar puncture between one of the L3 and L4 vertebrae and the L4 and L5 vertebrae.

5. The method of claim 1, wherein the step of injecting a volume is performed by a peristaltic pump.

6. The method of claim 1, wherein the step of gaining access to the subarachnoid space includes a cervical puncture.

7. The method of claim 1, wherein the distal tip of the aspiration catheter includes a radiopaque marker and the step of positioning the distal tip of the catheter proximate the obstruction is aided by fluoroscopy.

8. The method of claim 1, wherein the step of navigating the catheter through the ventricles includes passing the catheter through the cerebral aqueduct and accessing the third ventricle.

9. The method of claim 8, wherein the step of navigating the catheter through the ventricles further includes passing the catheter through the foramen of Monro and accessing one of the left or right lateral ventricle.

10. The method of claim 1, wherein the step of injecting the fluid to break-up the obstruction includes pulsating the fluid from the distal opening of the first lumen.

11. A method of re-circulating cerebral spinal fluid within a treatment area in the cranium, comprising the steps of:
providing a constant volume aspiration catheter operatively connected to a peristaltic pump and having a closed fluid circuit;
gaining access to the subarachnoid space of the spinal column;
navigating the aspiration catheter through the subarachnoid space to the base of the skull;
navigating the aspiration catheter along the brain in one of the subarachnoid space and ventricles until the treatment area is reached;
positioning a distal tip of the aspiration catheter proximate the treatment area within the cranium; and
operating the peristaltic pump to permit a volume of cerebrospinal fluid to be removed from the treatment area through an aspiration lumen of the aspiration catheter and to permit a volume of cerebrospinal fluid to be reintroduced through an injection lumen of the aspiration catheter into the treatment area, wherein the removed volume of cerebral spinal fluid substantially equals the reintroduced volume of cerebral spinal fluid such that the peristaltic pump maintains a constant volume of cerebrospinal fluid within the treatment area.

12. The method of claim 11, wherein the treatment area is within the brain.

13. The method of claim 12, wherein the steps of navigating the aspiration catheter includes entering the fourth ventricle of the brain via one of the foramen of Magendie and the foramina of Luschka and navigating through the ventricles of the brain until the treatment area is reached.

14. The method of claim 11, wherein the treatment area is within the subarachnoid space within the cranium.

15. The method of claim 14, wherein the steps of navigating the aspiration catheter include navigating the aspiration catheter in the subarachnoid space proximate the brain to the treatment site.

16. The method of claim 11, wherein the removed volume of cerebral spinal fluid passes through a filtering device prior to being reintroduced into the treatment area.

17. A method of removing and replacing cerebral spinal fluid within a treatment area during a ventriculostomy, comprising the steps of:
initiating a ventriculostomy within the brain;
providing a constant volume aspiration catheter operatively connected to a peristaltic pump for use during the ventriculostomy;
operating the peristaltic pump to permit a volume of cerebrospinal fluid to be removed from the ventriculostomy treatment area through an aspiration lumen of the aspiration catheter and to permit a volume of cerebrospinal fluid to be reintroduced through an injection lumen of the aspiration catheter into the treatment area, wherein the removed volume of cerebral spinal fluid substantially equals the reintroduced volume of cerebral spinal fluid such that the peristaltic pump maintains a constant volume of cerebrospinal fluid within the treatment area.

18. The method of claim 17, wherein the removed volume of cerebral spinal fluid passes through a filtering device prior to being reintroduced into the treatment area.

19. The method of claim 17, wherein the removed volume of cerebral spinal fluid is discarded and the volume of cerebrospinal fluid to be reintroduced is one of saline and artificial cerebral spinal fluid.

20. The method of claim 17, wherein the ventriculostomy is an endoscopic third ventriculostomy.
